(19) United States
(12) Patent Application Publication
(10) Pub. No.: US 2026/0066063 A1
(43) Pub. Date: Mar. 5, 2026

(54) SYSTEM AND METHOD FOR SELECTING BI-METALLIC METAL ORGANIC FRAMEWORK BASED ON PREDICTED PROPERTIES

(71) Applicant: Accenture Global Solutions Limited, Dublin (IE)

(72) Inventors: Shubhashis SENGUPTA, Bangalore (IN); Ravi Kumar Dhar, Mumbai (IN); Anandu Koikkalethu Balachandran, Pathanamthitta (IN); Lakshmi Priya Krishnamurthy, Trichy (IN)

(21) Appl. No.: 18/821,996

(22) Filed: Aug. 30, 2024

(51) Int. Cl. G16C 60/00; G16C 20/30; G16C 20/70

(52) U.S. Cl. CPC G16C 60/00; G16C 20/30; G16C 20/70

(57) ABSTRACT

Method, apparatus, system, and/or non-transitory computer readable media for creating a bi-metallic metal organic framework, which receives data representative of desired metal types and one or more linker functionality; generates a plurality of bi-metallic metal organic framework structures; extracts a machine-readable format of the plurality of bi-metallic metal organic framework structures; extracts relevant atomic features from the machine-readable format of the plurality of bi-metallic metal organic framework structures; predicts properties relative to the desired metal types and the one or more linker functionality, of the plurality of bi-metallic metal organic framework structures; and selects one or more of the plurality of bi-metallic metal organic framework structures.

### TECHNICAL FIELD

Various embodiments described herein relate generally to a computer-implemented method, a computer system, and a computer program product for creating a bi-metallic metal organic framework.

### BACKGROUND

Generative Artificial Intelligence (Gen AI) refers to advanced AI systems that emulate human cognitive abilities across various applications. These advanced AI systems use sophisticated methods to autonomously process complex data, make decisions, and solve problems. Further, Gen AI encompasses a broad category of AI systems, including specialized subsets like Large Language Models (LLMs) designed for Natural Language Processing (NLP) tasks. The LLMs are trained to understand and generate human-like responses based on input prompts. The LLMs excel in tasks such as language translation, text summarization, sentiment analysis, contextual understanding, and the like.

Metal organic frameworks (MOFs) can be constructed from inorganic nodes with organic linkers. MOFs include two heterometallic ions bonded to an organic ligand.

### BRIEF DESCRIPTION OF DRAWINGS

Various embodiments in accordance with the present disclosure will be described with reference to the drawings, in which:

FIG. 1 illustrates an example environment that may be used to execute implementations of the present disclosure.

FIG. 2 illustrates a flow chart illustrating an example flow according to at least one implementation of the present disclosure.

FIG. 3 illustrates a flow chart illustrating another example flow according to at least one implementation of the present disclosure.

FIG. 4 illustrates a computer system that may be used to implement a cache management system.

Like reference numbers and designations in the various drawings indicate like elements.

### DETAILED DESCRIPTION

In the following description, various embodiments will be illustrated by way of example and not by way of limitation in the figures of the accompanying drawings. References to various embodiments in this disclosure are not necessarily to the same embodiment, and such references mean at least one. While specific implementations and other details are discussed, it is to be understood that this is done for illustrative purposes only. A person skilled in the relevant art will recognize that other components and configurations may be used without departing from the scope of the claimed subject matter.

Reference to any “example” herein (e.g., “for example,” “an example of,” by way of an example” or the like) are to be considered non-limiting examples regardless of whether expressly stated or not.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

Without intent to limit the scope of the disclosure, examples of instruments, apparatus, methods, and their related results according to the embodiments of the present disclosure are given below. Note that titles or subtitles may be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

The term “comprising” when utilized means “including, but not necessarily limited to;” it specifically indicates open-ended inclusion or membership in the so-described combination, group, series, and the like.

The term “a” means “one or more” unless the context clearly indicates a single element.

“First,” “second,” etc., are labels to distinguish components or blocks of otherwise similar names but does not imply any sequence or numerical limitation.

“And/or” for two possibilities means either or both of the stated possibilities (“A and/or B” covers A alone, B alone, or both A and B take together), and when present with three or more stated possibilities means any individual possibility alone, all possibilities taken together, or some combination of possibilities that is less than all of the possibilities. The language in the format “at least one of A . . . and N” where A through N are possibilities means “and/or” for the stated possibilities (e.g., at least one A, at least one N, at least one A and at least one N, etc.).

It should also be noted that in some alternative implementations, the functions/acts noted may occur out of the order noted in the figures. For example, two steps disclosed or shown in succession may in fact be executed substantially concurrently or may sometimes be executed in the reverse order, depending upon the functionality/acts involved.

Specific details are provided in the following description to provide a thorough understanding of embodiments. However, it will be understood by one of ordinary skill in the art that embodiments may be practiced without these specific details. For example, systems may be shown in block diagrams so as not to obscure the embodiments in unnecessary detail. In other instances, well-known processes, structures, and techniques may be shown without unnecessary detail in order to avoid obscuring example embodiments.

The specification and drawings are to be regarded in an illustrative rather than a restrictive sense. It will, however, be evident that various modifications and changes may be made thereunto without departing from the broader spirit and scope of the disclosure as set forth in the claims.

With the advent of Generative Artificial Intelligence (Gen AI) systems, enterprises are adopting the Gen AI systems to support execution of various tasks/processes. For example, a Gen AI system may support communications and interactions, and processes in software systems to support decision-making within the enterprises. Multiple applications within a corporate network environment may use and interact with Large Language Models (LLMs) of the Gen AI systems to provide input and/or data for the execution of a wide variety of tasks, such as, human computer interactions (e.g., question and answering), automating process execution, process planning, generating step-by-step procedures for the process execution, performing data analysis, and/or the like. The LLMs operate by processing inputs to generate coherent, and contextually appropriate responses.

The present disclosure provides methods, systems, and apparatuses that are configured to create a bi-metallic metal organic framework. Designing bi-metallic MOFs inside a lab is very difficult because of the labor and interactions that are required. The present disclosure implements a targeted method that implements computational chemistry. The present disclosure also provides for the creation of materials using inverse design. The present disclosure generates material structures to satisfy possible rare or even conflicting target property constraints using generative models, evolutionary algorithms, and/or reinforcement learning.

The present disclosure implements an AI approach for designed, generating, validating, and/or predicting novel bi-metallic MOFs. In at least one example the bi-metallic MOFs can be focused on carbon dioxide capture and conversion. The creating of bi-metallic MOF structures is implemented using MatterGen or other similar generative AI models. The generated structures are processed into a fine-tuned porous materials transformer model (PMT) for predicting physical and chemical properties of the bi-metallic MOFs. The PMT may predict key properties including adsorption capacity, catalytic activity, thermos-stability, and/or other physical and/or chemical characteristics for selection from a plurality of bi-metallic MOF structures. The present disclosure presents an integrated approach to the design and validation of bi-metallic MOFs.

The present disclosure may have one or more of the following advantages over traditional methods for designing bi-metallic MOFs, including for carbon dioxide capture and conversion:

Efficiency: The use of AI and ML techniques significantly accelerates the MOF design process compared to traditional trial-and-error methods by three orders of magnitude

Targeted Design: The ability to specify desired metal types and linker functionalities facilitates the generation of MOFs with tailored properties for CO2 conversion and resynthesis into useful chemicals.

In Silico Screening: The computational predictions from the PMT enable the efficient screening of a large number of MOF candidates, reducing the need for extensive experimental testing in the labs.

Iterative Improvement: The use of experimental validation data allows for continuous improvement of the design process through refinement of the AI models and the fine-tuned PMT.

The present disclosure includes a method, apparatus, system, and/or non-transitory computer readable media for creating a bi-metallic metal organic framework, which receives data representative of desired metal types and one or more linker functionality; generates a plurality of bi-metallic metal organic framework structures; extracts a machine-readable format of the plurality of bi-metallic metal organic framework structures; extracts relevant atomic features from the machine-readable format of the plurality of bi-metallic metal organic framework structures; predicts properties relative to the desired metal types and the one or more linker functionality, of the plurality of bi-metallic metal organic framework structures; and selects one or more of the plurality of bi-metallic metal organic framework structures.

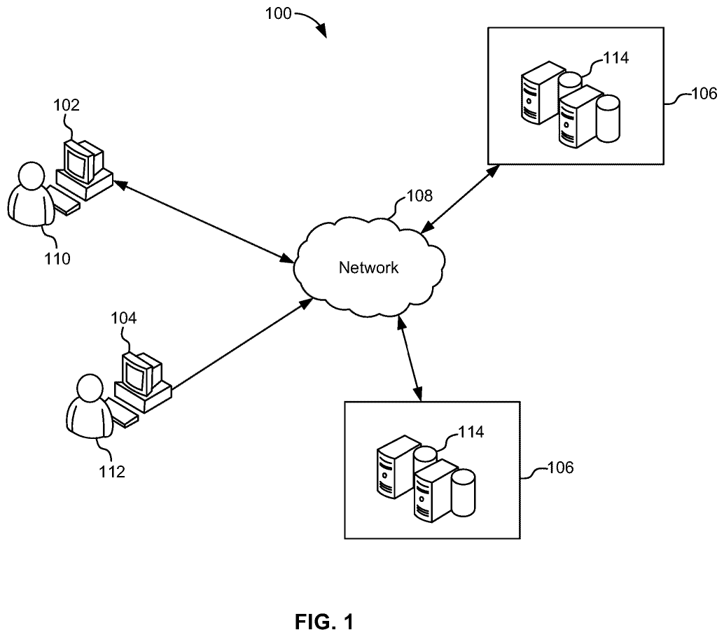

FIG. 1 illustrates an example environment 100 that may be used to execute implementations of the present disclosure. In some examples, the example environment 100 enables adaptive caching of responses generated by Large Language Models (LLMs).

As depicted in FIG. 1, the example environment 100 includes computing devices 102 and 104, back-end systems 106, and a network 108. In some examples, the computing devices 102 and 104 are used by respective users 110 and 112 to log into and interact with computing platforms executing applications according to implementations of the present disclosure. Examples of the computing devices 102 and 104 may include a server, a notebook, a desktop, a netbook, smartphones, laptops, a tablet, and/or voice-enabled devices. It is contemplated that implementations of the present disclosure may be realized with any appropriate type of computing device. In some examples, each of the computing devices 102 and 104 may include a web browser application executed thereon, which may be used to display one or more web pages of a computing platform executing applications. In some examples, each of the computing devices 102 and 104 may display one or more Graphical User Interfaces (GUIs) that enable the respective users 110 and 112 to interact with the computing platform.

In some examples, the network 108 may correspond to a communication network.

Examples of the network 108 may include, but are not limited to, a Local Area Network (LAN), a Wide Area Network (WAN), the Internet, Wi-Fi, Long Term Evolution (LTE), Worldwide Interoperability for Microwave Access (WiMAX), General Packet Radio Services (GPRS), or a combination thereof. The network 108 communicatively couples or connects the computing devices 102 and 104 with the back-end systems 106. In some examples, the network 108 may be accessed over a wired and/or a wireless communication link. For example, a computing device like smartphone may utilize a cellular network to access the network 108.

In some examples, one or more of the back-end systems 106 may be implemented as an on-premises system that is operated by an enterprise or a third-party engaged in cross-platform interactions and data management. In some examples, the back-end systems 106 may be implemented as an off-premises system (for example, a cloud or an on-demand system) that is operated by an enterprise or a third-party on behalf of an enterprise. In some examples, the back-end systems 106 may be implemented in a cloud environment. For simplicity, the back-end systems 106 depicted in FIG. 1 may be a cloud environment that is intended to represent various forms of servers including a web server, an application server, a proxy server, a network server, a server pool, and/or the like.

In some examples, each of the back-end systems 106 includes one or more cache management systems 114. A cache management system 114 may host components of enterprise systems and applications. Also, the cache management system 114 accepts requests from the users 110 and 112 through the respective computing devices 102 and 104 for services being provided by the enterprise systems and the applications. The requests received from the users 110 and 112 through the respective computing devices 102 and 104 may be prompts for one or more tasks. Examples of the tasks may include question-answering, automation of process execution, process planning, generation of step-by-step procedures, performing of data analysis.

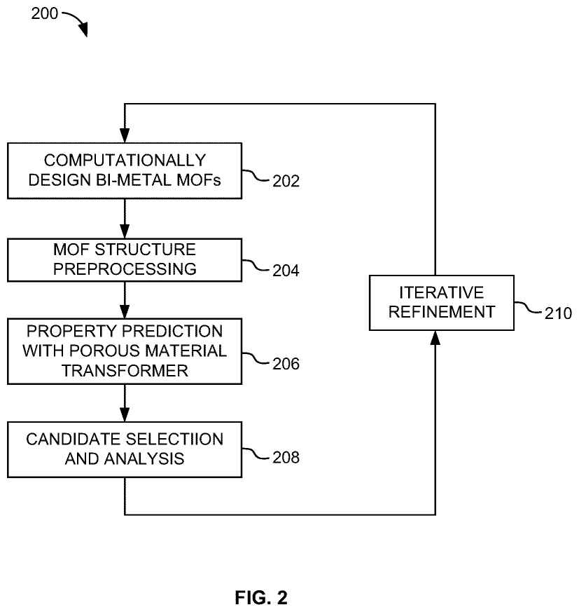

FIG. 2 illustrates an example flowchart 200 according to at least one implementation of the present disclosure. At block 202, the system can receive a request to design bi-metal metal organic framework(s) (MOFs). The system can implement a generative AI model to create a plurality of bi-metallic MOF structures. In at least one example, the generative AI can be MatterGen. In other examples, the generative AI can be used that target one or more properties. In at least one example, the properties can include desired metal types and linker functionalities. An example of desired metal types can include a specific metal or a plurality of metals. In order to illustrated, the present implementation, the example given throughout is focused on carbon capture and/or resynthesis. For example, the system can receive a request and/or prompt to consider a desired metal type of coper and/or nickel. Additionally, the system can receive a request and/or prompt for a linker functionalities related to carbon dioxide interaction. The system can receive the request and/or prompt and design bi-metallic MOF structures such that coper is designed to capture carbon dioxide and nickel is used to convert carbon dioxide into ethylene and/or methanol. The generative AI can leverage a combination of graph convolutional neural networks (GCNs) and variational autoencorders (VAEs) to generate novel MOF structures with the requested properties.

The generative AI can include a component that defines a chemical system and prepares input data. In at least one example, the generative AI may identify two metals that could potentially impart the desired characteristics to the MOF. The choice of metals may consider factors like oxidation state compatibility and coordination preferences. Additionally, in at least one example, the generative AI may gather data on various combinations of two metals with different organic linkers, highlighting successful coordination environments and stable configurations. Furthermore, the generative AI may label the data with relevant properties such as lattice parameters, space groups, and other pertinent physicochemical properties. Labelling allows the fine-tuning process to focus on specific characteristics which needs to be optimized.

The generative AI can also include integration of adapter modules. In at least one example, the adapter architecture may include adapter modules into a neural network architecture. The adapter modules may be tunable components that get inserted into specific layers of the main neural network architecture. The adapter modules may modify the behavior of the network in a controlled and targeted manner without the need to retrain the entire network. The adapter modules may provide for applications where computational resources are limited, or where the base model is complex and difficult to train.

The generative AI may also include conditioning outputs on specific target properties. The generative AI may receive data about the target properties (e.g., porosity or gas adsorption capacity in MOFs) and adjust the generation process at various points in the neural network. The adjustment may involve modifying the activation functions, adding bias terms, and/or scaling the outputs of certain layers in the network based on the input properties. E.g. if the target property is high gas adsorption capacity, the adapter might adjust the network to features known to enhance this property, such as increased surface area or optimal pore size distribution.

At block 204, the system may implement a MOF structure preprocessing. The system may convert the generated MOF structures into a machine-readable format (graph or sequence) compatible with a selected PMT. The system may extract relevant atomic features from the MOF structures for property prediction. The atomic features can include: geometric features, including but not limited to bond lengths (l_ij), angles (θ_ijk), dihedral angles (φ_ijk), pore size distribution (PSD); elemental features, including but not limited to types of metal atoms (M_i) and linker elements (X_j); and/or connectivity features, including but not limited to information about how atoms are connected within the MOF framework (adjacency matrix).

At block 206, the system may implement a property prediction with a porous material transformer (PMT). The PMT may be configured for different types of metals, and the one or more linker functionality. In at least one example, the system may pass the pre-processed MOF structures through a pre-trained PMT specifically designed for bi-metallic MOF's. The system may fine-tune the PMT on a curated dataset of bi-metallic MOFs with experimentally determined properties.

The PMT utilizes a transformer architecture to learn complex relationships between the MOF structure (encoded features) and desired properties. In at least one example, a desired property/feature can include carbon dioxide adsorption capacity, which may be predicted by the PMT based on the void fraction (V_void) within the MOF structure and the interaction energy (E_int) between carbon dioxide molecules and the metal centers. In one example, the system may use the following equation: carbon dioxide uptake≈V_void*f(E_int).

Additionally, the system can include catalytic activity, which may be inferred from the electronic properties of the bi-metallic MOF (derived from the generative AI) and its ability to bind and activate carbon dioxide molecules for conversion, which may be approximated by descriptors like the lowest unoccupied molecular orbital (LUMO) energy (ε_LUMO) and the band gap (ΔE). Higher ΔE and lower ε_LUMO can indicate better carbon dioxide activation potential. The system may include other physical and/or chemical properties, which the PMT can also predict properties like thermal stability (calculated from vibrational frequencies), mechanical stability (estimated from bond dissociation energies), and surface area (derived from pore size distribution).

The PMT may include tunable portions and optimization algorithm. The system is implemented to build towards universal transfer learning, both pre-training and fine-tuning strategies. Objective of pre-training may allow the PMT to learn the essential characteristics of a MOF. The pre-trained model serves as a starting point for all subsequent applications. Fine-tuning refers to the process of training the pre-trained models for the specific application such as the carbon dioxide uptake prediction, as described herein. PMT may capture two disparate types of features for MOFs: (1) local features (e.g., specific bonds and chemistry makeup of the building blocks) and/or (2) global features (e.g., geometric and topological descriptors).

The tunable portions may include a proper prediction with a decoder. The decoder component of the transformer utilizes the encoded information from the linker molecule to predict the desired MOF properties. One or more optional features include regression models, which can be implemented to predict continuous properties such as carbon dioxide uptake capacity based on learned relationships between linker structure and existing MOF data. An example of a linker structure can be C6H3O12, which has relevant atomic positions and connectivity information.

The system can include simple deep neural network and/or artificial neural network (ANN) at the end of transformer may predict carbon dioxide uptake values. The system can have a plurality of dense layers in ANN followed by a single neuron at the end which will give predicted carbon dioxide update values. The plurality of dense layers can number two or more. In one example, the plurality of dense layers is limited to two or three. During pretraining, the models may understand the topological, geometrical descriptors such as pore volume, surface area and/or atomistic information of building blocks and specific bonds. In at least one example, the system may include classification models, which categorize properties like band gap (wide vs. narrow) based on patterns identified during pre-training, fine tuning with the MOF dataset.

In still other examples, Mean Squared Error (MSE) or Mean Absolute Error (MAE) may be implemented as a loss function for training. Additionally, the system may pretrain the PMT on a plurality of tasks including but not limited to Void Fraction Prediction (VFP), MOF Topology Prediction (MTP) and/or Catalytic Activity Prediction The loss function used during training determines how the model is penalized for errors in predicting the target properties. For MOF design, a combination of loss functions can be employed: Mean Squared Error (MSE): For properties like CO2 uptake (continuous value) and/or binary cross-entropy. In one example, the MSE can be given by the following formula:

MSE=N1i=1ΣN(yi−y\hat{ }i)2 where: (N) is the number of data points; (y_i) represents the actual value; (\hat{y}_i) represents the predicted value.

Binary cross-entropy, which may be used for classifying catalytic activity towards specific products (e.g., ethylene vs. methanol) may include the following formula: Binary Cross-Entropy=−N1i=1ΣN(yi log(y\hat{ }i)+(1−yi)log(1−y\hat{ }i)) where: (N) is the number of data points; (y_i) is the true label (0 or 1); and (\hat{y}_i) is the predicted probability.

The tunable portions may also include an optimization algorithm. The PMT training process utilizes an optimization algorithm like Adam (Adaptive Moment Estimation) to update the weights and biases within the transformer network. Adam combines the benefits of gradient descent with momentum and adaptive learning rates, leading to faster convergence and improved model performance.

At block 208, the system may perform candidate selection and analysis. The system may analyze the predicted properties (for example, carbon dioxide uptake and catalytic activity) from the PMT to identify promising MOF candidates for further evaluation. The system may utilize visualization tools to explore the structural features of the predicted MOFs and correlate them with the predicted properties. Techniques may include crystal structure visualization software used to analyze pore size distribution and the arrangement of metal centers within the MOF framework. The system may employ statistical analysis techniques to identify trends and patterns within the generated MOF candidates. In at least one example, techniques like clustering algorithms may be implemented to group MOFs with similar properties and dimensionality reduction methods to visualize relationships between various features.

At block 210, the system may include iterative refinement. The MOF design process may include one or more iterative cycles. Data obtained from experimental validation may be used to further refine the generative AI model and the PMT. For example, MOFs with experimentally verified high carbon dioxide uptake and desired catalytic activity can be used to enrich the training data for the PMT, leading to improved prediction accuracy for future iterations.

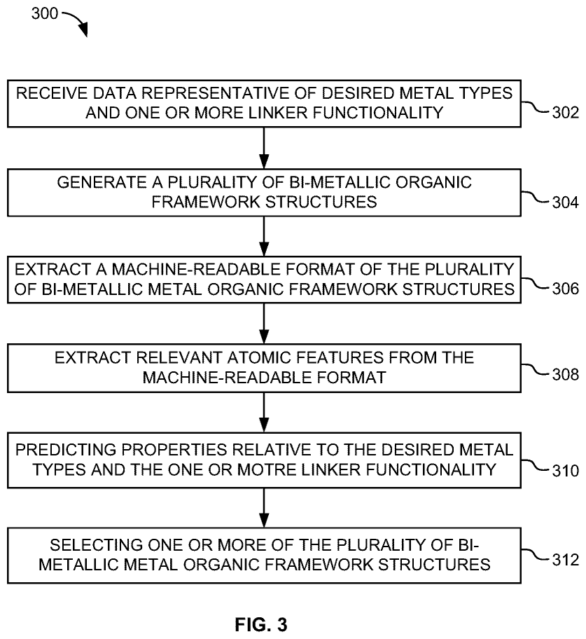

FIG. 3 illustrates a flow chart 300 according to the present disclosure for creating a bi-metallic metal organic framework. At block 302, the method includes receiving, at one or more processors, data representative of desired metal types and one or more linker functionality. In at least one example, the method may include receiving, at the one or more processors, an intended use of the bi-metallic metal organic framework; predicting properties related to the intended use; and selecting, based upon the intended use, one or more of the plurality of bi-metallic metal organic framework structures. In at least one example, the desired metal types include copper and nickel. The desired metal types can be selected using a prompt presented to a suer of the system. The desired metal types can be selected based on the user's knowledge of chemical reactiveness, or other known metal properties. Additionally, the method can include prompting for later selection of desired properties as described herein. For example, the method can include prompting for high catalytic activity for carbon dioxide conversion.

At block 304, the method includes generating, from a generative artificial intelligence model based upon the data, a plurality of bi-metallic metal organic framework structures. In at least one example, the generative artificial intelligence model implements a graph convolution neural network, operable to represent sub molecular structures, and a variational autoencoder architecture. In at least one example, the generative artificial intelligence model may include MatterGen.

At block 306, the method includes extracting, from the generative artificial intelligence model, a machine-readable format of the plurality of bi-metallic metal organic framework structures.

At block 308, the method includes extracting relevant atomic features from the machine-readable format of the plurality of bi-metallic metal organic framework structures.

At block 310, the method includes predicting properties, based on a porous material transformer, relative to the desired metal types and the one or more linker functionality, of the plurality of bi-metallic metal organic framework structures. The predicted properties may include gas adsorption capacity, specific surface area, catalytic activity, porosity, thermal stability, mechanical stability, and/or surface area. The method can consider a preselected number of the predicted properties as having enhanced weight depending upon a desired property set. For example, in the carbon capture implementation the porosity, gas adsorption capacity, and/or catalytic activity can be selected as the primary properties of interest and receive a higher weighting as compared to others of the predicted properties. The method may also include training the porous material transformer on synthetically generated dataset of bi-metallic metal organic frameworks. The method may also include refining the training based on an evaluation of the generated plurality of bi-metallic metal organic framework structures including adjusting loss functions to prioritize features or properties related to the desired property; and/or fine-tuning hyperparameters of the generative artificial intelligence model.

The porous material transformer (PMT) can be implemented using one or more of the following features. The generated linker molecules are processed through the PMT, a deep learning architecture specifically designed for porous materials. The PMT utilizes a transformer architecture with self-attention mechanisms. The self-attention mechanisms focus on crucial parts of the molecule structure (represented as mathematical vectors) during property prediction. To build towards universal transfer learning, both pre-training and fine-tuning strategies are implemented. An objective of pre-training is to allow the PMT to learn the essential characteristics of a MOF. This pre-trained model serves as a starting point for all subsequent applications. Fine-tuning refers to the process of training the pre-trained models for the specific application like CO2 uptake prediction. PMT captures two disparate types of features for MOFs: (1) local features (e.g., specific bonds and chemistry makeup of the building blocks) and (2) global features (e.g., geometric and topological descriptors).

The method can include a crossover method to bridge the gap been the generative artificial intelligence model and the PMT. The structures with specific applicants can be generated computationally and are then passed into the PMT to validate physical and chemical properties. The method can include a first stage and a second stage.

In the first stage, bi-metal compatibility filtering may occur. Linker molecules undergo a filtering step using custom algorithms to assess their suitability for incorporating two different metal ions within the MOF structure (e.g., coordination number calculations, distance geometry checks). Machine learning algorithms are employed to analyze the linker molecule's structure and identify potential metal binding sites. These algorithms can be trained on a dataset of existing MOF structures with known metal-linker interactions. Custom algorithms calculate the coordination number (CN) for each potential metal binding site. In at least one example, the calculation of the CN includes estimating interatomic distances between the metal ion and surrounding linker atoms; and/or employing empirical rules or established calculation methods like Sanderson's method to assess the feasibility of stable metal-linker bond formation based on ionic radii and bond length preferences.

In the second stage, targeted property prediction may occur. The second stage can include the linker molecule generated by generative artificial intelligence being converted into a machine-readable format suitable for the transformer architecture. The representation may include one or more of the following: i) graph representation with encoding the linker structure as a graph where nodes represent atoms and edges represent bonds between them; and/or ii) sequential representation with representing the linker as a sequence of atomic features like element type, connectivity, and spatial coordinates.

The second stage may also include transformer encoder processing. The PMT's encoder processes the linker molecule representation. The encoder may involve multiple layers of neural networks that analyze the structural information and extract relevant features for property prediction. The self-attention mechanism within the transformer allows the model to focus on crucial parts of the linker structure that significantly influence the target MOF properties (e.g., CO2 uptake sites).

The second stage may also include a property prediction with decoder. The decoder component of the transformer utilizes the encoded information from the linker molecule to predict the desired MOF properties. In one example, regression models are implemented to predict continuous properties like CO2 uptake capacity based on learned relationships between linker structure and existing MOF data.

At block 312, the method includes selecting, based on the predicted properties, one or more of the plurality of bi-metallic metal organic framework structures. In at least one example, the selecting is based on a greater than sixty percent match. In at least one example, the selecting is based on a greater than ninety-five percent match. Match as used herein refers to the predicted properties being within that percentage of desired properties. The method may include selecting a bi-metallic metal organic framework structure with a predicted high catalytic activity for carbon dioxide conversion.

The method may include analyzing the selected one or more of the plurality of bi-metallic metal organic framework structures using visualization tools and/or statistical analysis techniques.

Additionally, in at least one example, the method may include refining, implemented by one or more high-fidelity computational model, the predicted properties of the selected one or more of the plurality of bi-metallic metal organic framework structures.

In at least one example, the method may include synthesizing the selected one or more of the plurality of bi-metallic metal organic framework structures; and collecting measurement data on carbon dioxide conversion performance and/or catalytic activity of the synthesized one or more of the plurality of bi-metallic metal organic framework structures. The method may also include refining, the generative artificial intelligence model, based upon the measurement data; and tuning, the porous material transformer, based upon the measurement data.

The present disclosure also includes a non-transitory computer readable media storing instructions programed to cooperated with electronic computer hardware in combination with software to perform operations for creating a bi-metallic metal organic framework implementing one or more of the steps described herein. Furthermore, the present disclosure includes a system comprising at least one process and a non-transitory computer readable media as described herein.

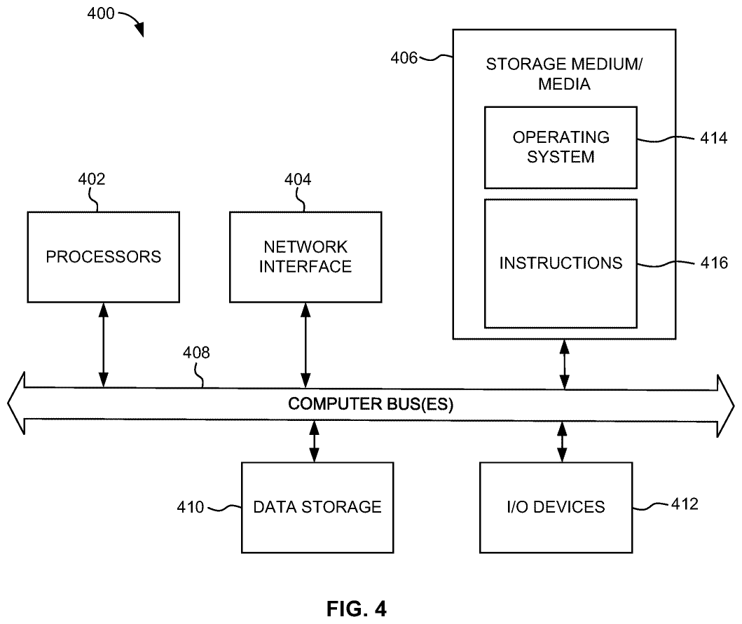

FIG. 4 illustrates a computer system 400 that may be used to implement creating a bi-metallic metal organic framework. More particularly, computing machines such as desktops, laptops, smartphones, tablets, and/or wearable electronic devices which may be used for adaptive caching of responses generated by the LLM and may have the structure of the computer system 400. The computer system 400 may include additional components not shown and that some of the process components described may be removed and/or modified. In another example, a computer system 400 may be deployed on external-cloud platforms such as cloud, internal corporate cloud computing clusters, organizational computing resources, and/or the like.

The computer system 400 includes processor(s) 402, such as a central processing unit, a controller, an application specific integrated circuit (ASIC), or another type of processing circuit, input/output devices (I/O) 404, such as a display, a mouse, a keyboard, etc., a network interface 406, such as a Local Area Network (LAN) interface, a wireless 802.11x interface, a 3G, 4G, 5G, or 6G mobile WAN or a WiMax WAN, and a processor-readable medium 408. Each of these components may be operatively coupled each other via one or more computer bus(es) 410. The computer-readable medium 408 may be any suitable medium that participates in providing instructions to the processor(s) 402 for execution. For example, the computer-readable medium 408 may be non-transitory or non-volatile medium, such as a magnetic disk or solid-state non-volatile memory or volatile medium such as RAM. The instructions or modules stored on the computer-readable medium 408 may include machine-readable or machine-executable instructions or code 412 executed by the processor(s) 402 that cause the processor(s) 402 to perform the methods and functions of creating a bi-metallic metal organic framework.

The creating a bi-metallic metal organic framework may be implemented as software stored on a non-transitory processor-readable medium and executed by the processors 402. For example, the computer-readable medium 408 may store an operating system 414, such as MAC OS, MS WINDOWS, UNIX, or LINUX, and code 412 for the creating a bi-metallic metal organic framework. The operating system 414 may be multi-user, multiprocessing, multitasking, multithreading, real-time, and the like. For example, during runtime, the operating system 414 and the code for the creating a bi-metallic metal organic framework are executed by the processor(s) 402.

The computer system 400 may include a data storage 416, which may include non-volatile data storage. The data storage 416 stores any data used or generated by the creating a bi-metallic metal organic framework.

The network interface 406 connects the computer system 400 to external systems for example, via a LAN. Also, the network interface 406 may connect the computer system 400 to the Internet. For example, the computer system 400 may connect to web browsers and other external applications and systems via the network interface 406.

What has been described and illustrated herein is an example along with some of its variations. The terms, descriptions, and figures used herein are set forth by way of illustration only and are not meant as limitations. Many variations are possible within the spirit and scope of the subject matter, which is intended to be defined by the following claims and their equivalents.

Implementations and all of the functional operations described in this specification may be realized in digital electronic circuitry, or in computer software, firmware, or hardware, including the structures disclosed in this specification and their structural equivalents, or in combinations of one or more of them. Implementations may be realized as one or more computer program products (e.g., one or more modules of computer program instructions encoded on a computer readable medium for execution by, or to control the operation of, data processing apparatus). The computer readable medium may be a machine-readable storage device, a machine-readable storage substrate, a memory device, a composition of matter effecting a machine-readable propagated signal, or a combination of one or more of them. The term computing system encompasses all apparatus, devices, and machines for processing data, including by way of example a programmable processor, a computer, or multiple processors or computers. The apparatus may include, in addition to hardware, code that creates an execution environment for the computer program in question (e.g., code that constitutes processor firmware, a protocol stack, a database management system, an operating system, or any appropriate combination of one or more thereof). A propagated signal is an artificially generated signal (e.g., a machine-generated electrical, optical, or electromagnetic signal) that is generated to encode information for transmission to suitable receiver apparatus.

A computer program (also known as a program, software, software application, script, or code) may be written in any appropriate form of programming language, including compiled or interpreted languages, and it may be deployed in any appropriate form, including as a stand-alone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform functions by operating on input data and generating output. The processes and logic flows may also be performed by, and apparatus may also be implemented as, special purpose logic circuitry (e.g., an FPGA (field programmable gate array) or an ASIC (application specific integrated circuit)).

Processors suitable for the execution of a computer program include, by way of example, both general and special purpose microprocessors, and any one or more processors of any appropriate kind of digital computer. Generally, a processor will receive instructions and data from a read only memory or a random-access memory or both. Elements of a computer can include a processor for performing instructions and one or more memory devices for storing instructions and data. Generally, a computer will also include, or be operatively coupled to receive data from or transfer data to, or both, one or more mass storage devices for storing data (e.g., magnetic, magneto optical disks, or optical disks). However, a computer need not have such devices. Moreover, a computer may be embedded in another device (e.g., a mobile telephone, a personal digital assistant (PDA), a mobile audio player, a Global Positioning System (GPS) receiver). Computer readable media suitable for storing computer program instructions and data include all forms of non-volatile memory, media and memory devices, including by way of example semiconductor memory devices (e.g., EPROM, EEPROM, and flash memory devices); magnetic disks (e.g., internal hard disks or removable disks); magneto optical disks; and CD ROM and DVD-ROM disks. The processor and the memory may be supplemented by, or incorporated in, special purpose logic circuitry.

To provide for interaction with a user, implementations may be realized on a computer having a display device (e.g., a CRT (cathode ray tube), LCD (liquid crystal display) monitor) for displaying information to the user and a keyboard and a pointing device (e.g., a mouse, a trackball, a touch-pad), by which the user may provide input to the computer. Other kinds of devices may be used to provide for interaction with a user as well; for example, feedback provided to the user may be any appropriate form of sensory feedback (e.g., visual feedback, auditory feedback, tactile feedback); and input from the user may be received in any appropriate form, including acoustic, speech, or tactile input.

Implementations may be realized in a computing system that includes a back end component (e.g., as a data server), a middleware component (e.g., an application server), and/or a front end component (e.g., a client computer having a graphical user interface or a Web browser, through which a user may interact with an implementation), or any appropriate combination of one or more such back end, middleware, or front end components. The components of the system may be interconnected by any appropriate form or medium of digital data communication (e.g., a communication network). Examples of communication networks include a local area network (LAN) and a wide area network (WAN), e.g., the Internet.

The computing system may include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of the disclosure or of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

A number of implementations have been described. Nevertheless, it will be understood that various modifications may be made without departing from the spirit and scope of the disclosure. For example, various forms of the flows shown above may be used, with steps re-ordered, added, or removed. Accordingly, other implementations are within the scope of the following claims.

What is claimed is:

1. A method for creating a bi-metallic metal organic framework comprising:
receiving, at one or more processors, data representative of desired metal types and one or more linker functionality;
generating, from a generative artificial intelligence model based upon the data, a plurality of bi-metallic metal organic framework structures;
extracting, from the generative artificial intelligence model, a machine-readable format of the plurality of bi-metallic metal organic framework structures;
extracting relevant atomic features from the machine-readable format of the plurality of bi-metallic metal organic framework structures;
predicting properties, based on a porous material transformer, relative to the desired metal types and the one or more linker functionality, of the plurality of bi-metallic metal organic framework structures; and
selecting, based on the predicted properties, one or more of the plurality of bi-metallic metal organic framework structures.

2. The method of claim 1, wherein the selecting is based on a greater than sixty percent match.

3. The method of claim 1, wherein the selecting is based on a greater than ninety-five percent match.

4. The method of claim 1, further comprising receiving, at the one or more processors, an intended use of the bi-metallic metal organic framework; predicting properties related to the intended use; and selecting, based upon the intended use, one or more of the plurality of bi-metallic metal organic framework structures.

5. The method of claim 1, wherein the generative artificial intelligence model implements a graph convolution neural network, operable to represent sub molecular structures, and a variational autoencoder architecture.

6. The method of claim 1, wherein the predicted properties include gas adsorption capacity, specific surface area, catalytic activity, porosity, thermal stability, mechanical stability, and/or surface area.

7. The method of claim 1, further comprising:
analyzing the selected one or more of the plurality of bi-metallic metal organic framework structures using visualization tools and/or statistical analysis techniques.

8. The method of claim 1, further comprising:
refining, implemented by one or more high-fidelity computational model, the predicted properties of the selected one or more of the plurality of bi-metallic metal organic framework structures.

9. The method of claim 1, further comprising:
synthesizing the selected one or more of the plurality of bi-metallic metal organic framework structures; and
collecting measurement data on carbon dioxide conversion performance and/or catalytic activity of the synthesized one or more of the plurality of bi-metallic metal organic framework structures.

10. The method of claim 9, further comprising:
refining, the generative artificial intelligence model, based upon the measurement data; and
tuning, the porous material transformer, based upon the measurement data.

11. The method of claim 1, further comprising: training the porous material transformer on synthetically generated dataset of bi-metallic metal organic frameworks.

12. The method of claim 11, further comprising: refining the training based on an evaluation of the generated plurality of bi-metallic metal organic framework structures including adjusting loss functions to prioritize features or properties related to the desired property; and/or fine-tuning hyperparameters of the generative artificial intelligence model.

13. The method of claim 1, wherein the desired metal types include copper and nickel.

14. The method of claim 11, further comprising selecting a bi-metallic metal organic framework structure with a predicted high catalytic activity for carbon dioxide conversion.

15. A non-transitory computer readable media storing instructions programmed to cooperate with electronic computer hardware in combination with software to perform operations for creating a bi-metallic metal organic framework, the operations comprising:
receive data representative of desired metal types and one or more linker functionality;
generate, from a generative artificial intelligence model based upon the data, a plurality of bi-metallic metal organic framework structures;
extract, from the generative artificial intelligence model, a machine-readable format of the plurality of bi-metallic metal organic framework structures;
extract relevant atomic features from the machine-readable format of the plurality of bi-metallic metal organic framework structures;
predict properties, based on a porous material transformer, relative to the desired metal types and the one or more linker functionality, of the plurality of bi-metallic metal organic framework structures; and
select, based on the predicted properties, one or more of the plurality of bi-metallic metal organic framework structures.

16. The non-transitory computer readable media of claim 15, further comprising:
synthesizing the selected one or more of the plurality of bi-metallic metal organic framework structures; and
collecting measurement data on carbon dioxide conversion performance and/or catalytic activity of the synthesized one or more of the plurality of bi-metallic metal organic framework structures.

17. The non-transitory computer readable media of claim 16, further comprising:
refining, the generative artificial intelligence model, based upon the measurement data; and
tuning, the porous material transformer, based upon the measurement data.

18. The non-transitory computer readable media of claim 15, further comprising: training the porous material transformer on synthetically generated dataset of bi-metallic metal organic frameworks.

19. The non-transitory computer readable media of claim 18, further comprising: refining the training based on an evaluation of the generated plurality of bi-metallic metal organic framework structures including adjusting loss functions to prioritize features or properties related to the desired property; and/or fine-tuning hyperparameters of the generative artificial intelligence model.

20. A system comprising:
a processor; and
a non-transitory computer readable media storing instructions programmed to cooperate with the processor to perform operations for creating a bi-metallic metal organic framework, the operations comprising:
receive data representative of desired metal types and one or more linker functionality;
generate, from a generative artificial intelligence model based upon the data, a plurality of bi-metallic metal organic framework structures;
extract, from the generative artificial intelligence model, a machine-readable format of the plurality of bi-metallic metal organic framework structures;
extract relevant atomic features from the machine-readable format of the plurality of bi-metallic metal organic framework structures;
predict properties, based on a porous material transformer, relative to the desired metal types and the one or more linker functionality, of the plurality of bi-metallic metal organic framework structures; and
select, based on the predicted properties, one or more of the plurality of bi-metallic metal organic framework structures.
